SEQUENCE LISTING (10 sequences)

SEQ ID NO: 1 - DNA, 906 bp, Danio rerio
  CDS (48)...(564)
agccagtgacccaggagcccagaccaacagcaaagcagacagtgaacatggctagtaaaaaagccgcggccaagagggggaaaactgctcaaagaggctcttccaatgtcttctccatgtttgagcaatcacaaatacaggagtttaaggaggcttttggctgcatagatcagaaccgggatggagttatcaacaaatctgatctgaaggaaacttatgcacaactagggaagctgaatgtgagtgatgaagagctggagtccatgctaacagaaggaaaagggcccataaacttcactgtcttcctcaccctctttggagagaagctcaatggcacagacccagaggaaaccatccttgctgcttttaaattgttcgaccctaatgccacaggagttgtcaataaagatgagttcaagaggctgctaatgacccaagctgataaattcacagcagaagaggttgaccaggcttttgcagtggctccaatagacgtggctggaaatattgattataagtctctttgttacattatcacacatggtgatgaaaaagaggaatcttgaagaaaggaaaatgtctcagtttatgtctgcatatctgttcagttcaacattcacatcagcagcttttaatattaaaacatttgtgttgcatttcaacacaagactaatgtctgttttatatacaaaataaacattagattacttgactgagcttaagagaagaccatgagaagagataattgtcatcttatttattctgtctgtaactgctgccccctacagactgttaatcgcaattaattactggacatttggtttaagggccttaaattcattgctatgagtagagcatcatctcaaacctataataaacttcatactcagttgctctcagaaaaaa

SEQ ID NO: 2 - protein, 172 aa, Danio rerio
MASKKAAAKRGKTAQRGSSNVFSMFEQSQIQEFKEAFGCIDQNRDGVINKSDLKETYAQLGKLNVSDEELESMLTEGKGPINFTVFLTLFGEKLNGTDPEETILAAFKLFDPNATGVVNKDEFKRLLMTQADKFTAEEVDQAFAVAPIDVAGNIDYKSLCYIITHGDEKEES

SEQ ID NO: 3 - DNA, 581 bp, Danio rerio
  CDS (80)...(523)
ggccagcaggaaggcggggacccggggcaaggtggcagccaccaagcaggcccaacgtggttcttccaacgtcttttccatgtttgaacaagcccagatacaggagttcaaagaagccttcagctgtatcgaccagaatcgtgatggcatcatctgcaaggcagacctgagggagacctactcccagctggggaaggtgagtgtcccagaggaggagctggacgccatgctgcaagagggcaagggccccatcaacttcaccgtcttcctcacgctctttggggagaagctcaatgggacagaccccgaggaagccatcctgagtgccttccgcatgtttgaccccagcggcaaaggggtggtgaacaaggatgagttcaagcagcttctcctgacccaggcagacaagttctctccagctgaggtggagcagatgttcgccctgacacccatggacctggcggggaacatcgactacaagtcactgtgctacatcatcacccatggagacgagaaagaggaatgaggggcagggccaggcccacgggggggcacctcaataaactctgttgcaaaattgg

SEQ ID NO: 4 - protein, 148 aa, Danio rerio
MFEQAQIQEFKEAFSCIDQNRDGIICKADLRETYSQLGKVSVPEEELDAMLQEGKGPINFTVFLTLFGEKLNGTDPEEAILSAFRMFDPSGKGVVNKDEFKQLLLTQADKFSPAEVEQMFALTPMDLAGNIDYKSLCYIITHGDEKEE

SEQ ID NO: 5 - DNA, 783 bp, Danio rerio
  CDS (1)...(783)
atggctagtaaaaaagccgcggccaagagggggaaaactgctcaaagaggctcttccaatgtcttctccatgtttgagcaatcacaaatacaggagtttaaggagggaagctgaatgtgagtgatgaagagctggagtccatgctaacagaaggaaaagggcccataaacttcactgtcttcctcaccctctttggagagaagctcaatggcacagacccagaggaaaccatccttgctgcttttaaattgttcgaccctaatgccacaggagttgtcaataaagatgagttcaagaggctgctaatgacccaagctgataaattcacagcagaagaggttgaccaggcttttgcagtggctccaatagacgtggctggaaatattgattataagtctctttgttacattatcacacatggtgatgaaaaagaggaatcttgaagaaaggaaaatgtctcagtttatgtctgcatatctgttcagttcaacattcacatcagcagcttttaatattaaaacatttgtgttgcatttcaacacaagactaatgtctgttttatatacaaaataaacattagattacttgactgagcttaagagaagaccatgagaagagataattgtcatcttatttattctgtctgtaactgctgccccctacagactgttaatcgcaattaattactggacatttggtttaagggccttaaattcattgctatgagtagagcatcatctcaaacctataataaacttcatactcagttgctctcagaaaaaa

SEQ ID NO: 6 - protein, 251 aa, Danio rerio
MASKKAAAKRGKTAQRGSSNVFSMFEQSQIQEFKEGSMVMKSWSPCQKEKGPTSLSSSPSLERSSMAQTQRKPSLLLLNCSTLMPQELSIKMSSRGCPKLINSQQKRLTRLLQWLQTWLEILIISLFVTLSHMVMKKRNLEERKMSQFMSAYLFSSTFTSAAFNIKTFVLHFNTRLMSVLYTKTLDYLTELKRRPEEIIVILFILSVTAAPYRLLIAINYWTFGLRALNSLLVEHHLKPIINFILSCSQKK

SEQ ID NO: 7 - DNA, 599 bp, Danio rerio
  CDS (1)...(597)
atggctagtaaaaaagccgcggccaagagggggaaaactgctcaaagaggctcttccaatgtcttctccatgtttgagcaatcacaaatacaggagtttaaggaggttcaagaggctgctaatgacccaagctgataaattcacagcagaagaggttgaccaggcttttgcagtggctccaatagacgtggctggaaatattgattataagtctctttgttacattatcacacatggtgatgaaaaagaggaatcttgaagaaaggaaaatgtctcagtttatgtctgcatatctgttcagttcaacattcacatcagcagcttttaatattaaaacatttgtgttgcatttcaacacaagactaatgtctgttttatatacaaaataaacattagattacttgactgagcttaagagaagaccatgagaagagataattgtcatcttatttattctgtctgtaactgctgccccctacagactgttaatcgcaattaattactggacatttggtttaagggccttaaattcattgctatgagtagagcatcatctcaaacctataataaacttcatactcagttgctctcagaaaaaa

SEQ ID NO: 8 - protein, 181 aa, Danio rerio
MASKKAAAKRGKTAQRGSSNVFSMFEQSQIQEFKEVQEAANDPSIHSRRGPGFCSGSNRRGWKYLVSLLHYHTWKRGILKKGKCLSLCLHICSVQHSHQQLLILKHLCCISTQDCLFYIQNKHITLSLREDHEKRLSSYLFCLLLPPTDCSQLITGHLVGPIHCYESIISNLTSYSVALRK

SEQ ID NO: 9 - DNA, 932 bp, Danio rerio
  CDS (1)...(930)
atggctagtaaaaaagccgcggccaagagggggaaaactgctcaaagaggctcttccaatgtcttctccatgtttgagcaatcacaaatacaggagtttaaggaggcttttggctgcatagatcagaaccgggatggagttatcaacaaatctgatctgaaggaaacttatgcacaactagatacacttgagactttcagaatgctttcatcatacatctagtgaaattataatgatatataatatgataccagggaagctgaatgtgagtgatgaagagctggagtccatgctaacagaaggaaaagggcccataaacttcactgtcttcctcaccctctttggagagaagctcaatggcacagacccagaggaaaccatccttgctgcttttaaattgttcgaccctaatgccacaggagttgtcaataaagatgagttcaagaggctgctaatgacccaagctgataaattcacagcagaagaggttgaccaggcttttgcagtggctccaatagacgtggctggaaatattgattataagtctctttgttacattatcacacatggtgatgaaaaagaggaatcttgaagaaaggaaaatgtctcagtttatgtctgcatatctgttcagttcaacattcacatcagcagcttttaatattaaaacatttgtgttgcatttcaacacaagactaatgtctgttttatatacaaaataaacattagattacttgactgagcttaagagaagaccatgagaagagataattgtcatcttatttattctgtctgtaactgctgccccctacagactgttaatcgcaattaattactggacatttggtttaagggccttaaattcattgctatgagtagagcatcatctcaaacctataataaacttcatactcagttgctctcagaaaaaa

SEQ ID NO: 10 - protein, 283 aa, Danio rerio
MASKKAAAKRGKTAQRGSSNVFSMFEQSQIQEFKEAFGCIDQNRDGVINKSDLKETYAQLDTLETFRMLSSYINYNDIYDTREAECERAGVHANRRKRAHKLHCLPHPLWREAQWHRPRGNHPCCFIVRPCHRSCQRVQEAANDPSIHSRRGPGFCSGSNRRGWKYLVSLLHYHTWKRGILKKGKCLSLCLHICSVQHSHQQLLILKHLCCISTQDCLFYIQNKHITLSLREDHEKRLSSYLFCLLLPPTDCSQLITGHLVGPIHCYESIISNLTSYSVALRK

(19) United States
(12) Patent Application Publication
(10) Pub. No.: US 2005/0064416 A1
(43) Pub. Date: Mar. 24, 2005

(54) Methods for diagnosing and treating heart disease

(76) Inventors: Mark C. Fishman, Newton Center, MA (US); Wolfgang Rottbauer, Regensburg (DE)

Correspondence Address: CLARK & ELBING LLP, 101 FEDERAL STREET, BOSTON, MA, 02110, US

(21) Appl. No.: 10/491,452

(22) PCT Filed: Oct. 1, 2002
(86) PCT No.: PCT/US02/31150

(30) Foreign Application Priority Data
Oct. 1, 2001 (US) 60326635

(51) Int. Cl. C12Q 1/68

(52) U.S. Cl. 435/6

(57) ABSTRACT

The invention provides methods of diagnosing and treating heart diseases and conditions, methods for identifying compounds that can be used to treat or to prevent such diseases and conditions, and methods of using these compounds to treat or to prevent such diseases and conditions. Also provided in the invention are animal model systems that can be used in screening methods.

### FIELD OF THE INVENTION

This invention relates to methods for diagnosing and treating heart disease.

### BACKGROUND OF THE INVENTION

Heart disease is a general term used to describe many different heart conditions. For example, coronary artery disease, which is the most common heart disease, is characterized by constriction or narrowing of the arteries supplying the heart with oxygen-rich blood, and can lead to myocardial infarction, which is the death of a portion of the heart muscle. Heart failure is a condition resulting from the inability of the heart to pump an adequate amount of blood through the body. Heart failure is not a sudden, abrupt stop of heart activity but, rather, typically develops slowly over many years, as the heart gradually loses its ability to pump blood efficiently. Risk factors for heart failure include coronary artery disease, hypertension, valvular heart disease, cardiomyopathy, disease of the heart muscle, obesity, diabetes, and a family history of heart failure.

The zebrafish, Danio rerio, is a convenient organism to use in genetic and biochemical analyses of development. It has an accessible and transparent embryo, allowing direct observation of organ function from the earliest stages of development, has a short generation time, and is fecund.

### SUMMARY OF THE INVENTION

The invention provides diagnostic, drug screening, and therapeutic methods that are based on the observation that a mutation, designated the “tell tale heart (tel)” mutation, in the zebrafish Myosin Light Chain 2a (MLC-2a) gene leads to a phenotype in zebrafish that is characterized by abnormal heart contractility.

In a first aspect, the invention provides a method of determining whether a test subject (e.g., a mammal, such as a human) has or is at risk of developing a disease or condition related to MLC-2a (e.g., a disease or condition of the heart, such as heart failure; also see below). This method involves analyzing a nucleic acid molecule of a sample from the test subject to determine whether the test subject has a mutation (e.g., the tel mutation; see below) in a gene encoding MLC-2a. The presence of such a mutation indicates that the test subject has or is at risk of developing a disease related to MLC-2a. This method can also involve the step of using nucleic acid molecule primers specific for a gene encoding MLC-2a for nucleic acid molecule amplification of the gene by the polymerase chain reaction. It can further involve sequencing a nucleic acid molecule encoding MLC-2a from a test subject.

In a second aspect, the invention provides a method for identifying compounds that can be used to treat or prevent a disease or condition associated with MLC-2a. This method involves contacting an organism (e.g., a zebrafish) having a mutation in a MLC-2a gene (e.g., the tell tale heart mutation), and having a phenotype characteristic of such a disease or condition, with the compound, and determining the effect of the compound on the phenotype. Detection of an improvement in the phenotype indicates the identification of a compound that can be used to treat or prevent the disease or condition.

In a third aspect, the invention provides a method of treating or preventing a disease or condition related to MLC-2a in a patient (e.g., a patient having a mutation (e.g., the tell tale heart mutation) in a MLC-2a gene), involving administering to the patient a compound identified using the method described above. Also included in the invention is the use of such compounds in the treatment or prevention of such diseases or conditions, as well as the use of these compounds in the preparation of medicaments for such treatment or prevention.

In a fourth aspect, the invention provides an additional method of treating or preventing a disease or condition related to MLC-2a in a patient. This method involves administering to the patient a functional MLC-2a protein or a nucleic acid molecule (in, e.g., an expression vector) encoding the protein. Also included in the invention is the use of such proteins or nucleic acid molecules in the treatment or prevention of such diseases or conditions, as well as the use of these proteins or nucleic acid molecules in the preparation of medicaments for such treatment or prevention.

In a fifth aspect, the invention includes a substantially pure zebrafish MLC-2a polypeptide. This polypeptide can include or consist essentially of, for example, an amino acid sequence that is substantially identical to the amino acid sequence of SEQ ID NO:2. The invention also includes variants of these polypeptides that include sequences that are at least 75%, 85%, or 95% (e.g., at least 91%, 92%, 93%, 94%, 95%, 96%, 97%, 98%, or 99%) identical to the sequences of these polypeptides, and which have MLC-2a activity or otherwise are characteristic of the diseases and conditions mentioned elsewhere herein. Fragments of these polypeptides are also included in the invention. For example, fragments that include any of the different domains of MLC-2a, in varying combinations, are included.

In a sixth aspect, the invention provides an isolated nucleic acid molecule (e.g., a DNA molecule) including a sequence encoding a zebrafish MLC-2a polypeptide. This nucleic acid molecule can encode a polypeptide including or consisting essentially of an amino sequence that is substantially identical to the amino acid sequence of SEQ ID NO:2. The invention also includes nucleic acid molecules that hybridize to the complement of SEQ ID NO:1 under highly stringent conditions and encode polypeptides that have MLC-2a activity or otherwise are characteristic of the diseases and conditions mentioned elsewhere herein.

In a seventh aspect, the invention provides a vector including the nucleic acid molecule described above.

In an eighth aspect, the invention includes a cell including the vector described above.

In a ninth aspect, the invention provides a non-human transgenic animal (e.g., a zebrafish or a mouse) including the nucleic acid molecule described above.

In a tenth aspect, the invention provides a non-human animal having a knockout mutation in one or both alleles encoding a MLC-2a polypeptide.

In an eleventh aspect, the invention includes a cell from the non-human knockout animal described above.

In a twelfth aspect, the invention includes a non-human transgenic animal (e.g., a zebrafish) including a nucleic acid molecule encoding a mutant MLC-2a polypeptide, e.g., a polypeptide having the tell tale heart mutation.

In a thirteenth aspect, the invention provides an antibody that specifically binds to a MLC-2a polypeptide.

In a fourteenth aspect, the invention provides a method of modulating the activity of a Myosin Light Chain 2a polypeptide in a patient, by administering to the patient an RNA that stimulates or inhibits this activity.

By “polypeptide” or “polypeptide fragment” is meant a chain of two or more (e.g., 10, 15, 20, 30, 50, 100, or 200, or more) amino acids, regardless of any post-translational modification (e.g., glycosylation or phosphorylation), constituting all or part of a naturally or non-naturally occurring polypeptide. By “post-translational modification” is meant any change to a polypeptide or polypeptide fragment during or after synthesis. Post-translational modifications can be produced naturally (such as during synthesis within a cell) or generated artificially (such as by recombinant or chemical means). A “protein” can be made up of one or more polypeptides.

By “Myosin Light Chain 2a protein,” “Myosin Light Chain 2a polypeptide,” “MLC-2a protein,” or “MLC-2a polypeptide” is meant a polypeptide that has at least 45%, preferably at least 60%, more preferably at least 75%, and most preferably at least 90% (e.g., at least 91%, 92%, 93%, 94%, 95%, 96%, 97%, 98%, or 99%) amino acid sequence identity to the sequence of a human (SEQ ID NO:4) or a zebrafish (SEQ ID NO:2) MLC-2a polypeptide. Polypeptide products from splice variants of MLC-2a gene sequences and MLC-2a genes containing mutations are also included in this definition. A MLC-2a polypeptide as defined herein plays a role in heart development, modeling, and function. It can be used as a marker of diseases and conditions associated with MLC-2a, such as heart disease (also see below).

By a “Myosin Light Chain 2a nucleic acid molecule” or “MLC-2a nucleic acid molecule” is meant a nucleic acid molecule, such as a genomic DNA, cDNA, or RNA (e.g., mRNA) molecule, that encodes a MLC-2a protein (e.g., a human (encoded by SEQ ID NO:3) or a zebrafish (encoded by SEQ ID NO:1) MLC-2a protein), a MLC-2a polypeptide, or a portion thereof, as defined above. A mutation in a MLC-2a nucleic acid molecule can be characterized, for example, by the insertion of a premature stop codon anywhere in the MLC-2a gene, or by a mutation in a splice donor site, which leads to aberrant transcript production (e.g., transcripts with premature stop codons (see, e.g., the description of the tel mutation, below)). In addition to tel and similar mutations, this zebrafish Myosin Light Chain 2a mutation (hereinafter referred to as “the tell tale heart mutation”), the invention includes any mutation that results in aberrant MLC-2a protein production or function, including, only as examples, null mutations and additional mutations causing truncations.

The term “identity” is used herein to describe the relationship of the sequence of a particular nucleic acid molecule or polypeptide to the sequence of a reference molecule of the same type. For example, if a polypeptide or a nucleic acid molecule has the same amino acid or nucleotide residue at a given position, compared to a reference molecule to which it is aligned, there is said to be “identity” at that position. The level of sequence identity of a nucleic acid molecule or a polypeptide to a reference molecule is typically measured using sequence analysis software with the default parameters specified therein, such as the introduction of gaps to achieve an optimal alignment (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). These software programs match identical or similar sequences by assigning degrees of identity to various substitutions, deletions, or other modifications. Conservative substitutions typically include substitutions within the following groups: glycine, alanine, valine, isoleucine, and leucine; aspartic acid, glutamic acid, asparagine, and glutamine; serine and threonine; lysine and arginine; and phenylalanine and tyro sine.

A nucleic acid molecule or polypeptide is said to be “substantially identical” to a reference molecule if it exhibits, over its entire length, at least 51%, preferably at least 55%, 60%, or 65%, and most preferably 75%, 85%, 90%, or 95% (e.g., at least 91%, 92%, 93%, 94%, 95%, 96%, 97%, 98%, or 99%) identity to the sequence of the reference molecule. For polypeptides, the length of comparison sequences is at least 16 amino acids, preferably at least 20 amino acids, more preferably at least 25 amino acids, and most preferably at least 35 amino acids. For nucleic acid molecules, the length of comparison sequences is at least 50 nucleotides, preferably at least 60 nucleotides, more preferably at least 75 nucleotides, and most preferably at least 110 nucleotides. Of course, the length of comparison can be any length up to and including full length.

A MLC-2a nucleic acid molecule or a MLC-2a polypeptide is “analyzed” or subject to “analysis” if a test procedure is carried out on it that allows the determination of its biological activity or whether it is wild type or mutated. For example, one can analyze the MLC-2a genes of an animal (e.g., a human or a zebrafish) by amplifying genomic DNA of the animal using the polymerase chain reaction, and then determining whether the amplified DNA contains a mutation, for example, the tell tale heart mutation, by, e.g., nucleotide sequence or restriction fragment analysis.

By “probe” or “primer” is meant a single-stranded DNA or RNA molecule of defined sequence that can base pair to a second DNA or RNA molecule that contains a complementary sequence (a “target”). The stability of the resulting hybrid depends upon the extent of the base pairing that occurs. This stability is affected by parameters such as the degree of complementarity between the probe and target molecule, and the degree of stringency of the hybridization conditions. The degree of hybridization stringency is affected by parameters such as the temperature, salt concentration, and concentration of organic molecules, such as formamide, and is determined by methods that are well known to those skilled in the art. Probes or primers specific for MLC-2a nucleic acid molecules, preferably, have greater than 45% sequence identity, more preferably at least 55-75% sequence identity, still more preferably at least 75-85% sequence identity, yet more preferably at least 85-99% sequence identity, and most preferably 100% sequence identity to the sequences of human (SEQ ID NO:3) or zebrafish (SEQ ID NO:1) MLC-2a genes.

Probes can be detectably labeled, either radioactively or non-radioactively, by methods that are well known to those skilled in the art. Probes can be used for methods involving nucleic acid hybridization, such as nucleic acid sequencing, nucleic acid amplification by the polymerase chain reaction, single stranded conformational polymorphism (SSCP) analysis, restriction fragment polymorphism (RFLP) analysis, Southern hybridization, northern hybridization, in situ hybridization, electrophoretic mobility shift assay (MSA), and other methods that are well known to those skilled in the art.

A molecule, e.g., an oligonucleotide probe or primer, a gene or fragment thereof, a cDNA molecule, a polypeptide, or an antibody, can be said to be “detectably-labeled” if it is marked in such a way that its presence can be directly identified in a sample. Methods for detectably labeling molecules are well known in the art and include, without limitation, radioactive labeling (e.g., with an isotope, such as ³²P or ³⁵S) and nonradioactive labeling (e.g., with a fluorescent label, such as fluorescein).

By a “substantially pure polypeptide” is meant a polypeptide (or a fragment thereof) that has been separated from proteins and organic molecules that naturally accompany it. Typically, a polypeptide is substantially pure when it is at least 60%, by weight, free from the proteins and naturally occurring organic molecules with which it is naturally associated. Preferably, the polypeptide is a MLC-2a polypeptide that is at least 75%, more preferably at least 90%, and most preferably at least 99%, by weight, pure. A substantially pure MLC-2a polypeptide can be obtained, for example, by extraction from a natural source, by expression of a recombinant nucleic acid molecule encoding a MLC-2a polypeptide, or by chemical synthesis. Purity can be measured by any appropriate method, e.g., by column chromatography, polyacrylamide gel electrophoresis, or HPLC analysis.

A polypeptide is substantially free of naturally associated components when it is separated from those proteins and organic molecules that accompany it in its natural state. Thus, a protein that is chemically synthesized or produced in a cellular system that is different from the cell in which it is naturally produced is substantially free from its naturally associated components. Accordingly, substantially pure polypeptides not only include those that are derived from eukaryotic organisms, but also those synthesized in E. coli, other prokaryotes, or in other such systems.

By “isolated nucleic acid molecule” is meant a nucleic acid molecule that is removed from the environment in which it naturally occurs. For example, a naturally-occurring nucleic acid molecule present in the genome of cell or as part of a gene bank is not isolated, but the same molecule, separated from the remaining part of the genome, as a result of, e.g., a cloning event (amplification), is “isolated.” Typically, an isolated nucleic acid molecule is free from nucleic acid regions (e.g., coding regions) with which it is immediately contiguous, at the 5′ or 3′ ends, in the naturally occurring genome. Such isolated nucleic acid molecules can be part of a vector or a composition and still be isolated, as such a vector or composition is not part of its natural environment.

An antibody is said to “specifically bind” to a polypeptide if it recognizes and binds to the polypeptide (e.g., a MLC-2a polypeptide), but does not substantially recognize and bind to other molecules (e.g., non-MLC-2a-related polypeptides) in a sample, e.g., a biological sample, which naturally includes the polypeptide.

By “high stringency conditions” is meant conditions that allow hybridization comparable with the hybridization that occurs using a DNA probe of at least 100, e.g., 200, 350, or 500, nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. (These are typical conditions for high stringency northern or Southern hybridizations.) High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually 16 nucleotides or longer for PCR or sequencing, and 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference.

By “sample” is meant a tissue biopsy, amniotic fluid, cell, blood, serum, urine, stool, or other specimen obtained from a patient or a test subject. The sample can be analyzed to detect a mutation in a MLC-2a gene, or expression levels of a MLC-2a gene, by methods that are known in the art. For example, methods such as sequencing, single-strand conformational polymorphism (SSCP) analysis, or restriction fragment length polymorphism (RFLP) analysis of PCR products derived from a patient sample can be used to detect a mutation in a MLC-2a gene; ELISA and other immunoassays can be used to measure levels of a MLC-2a polypeptide; and PCR can be used to measure the level of a MLC-2a nucleic acid molecule.

By “Tyosin Light Chain 2a-related disease,” “MLC-2a-related disease,” “Myosin Light Chain 2a-related condition,” or “MLC-2a-related condition” is meant a disease or condition that results from inappropriately high or low expression of a MLC-2a gene, or a mutation in a MLC-2a gene (including control sequences, such as promoters) that alters the biological activity of a MLC-2a nucleic acid molecule or polypeptide. MLC-2a-related diseases and conditions can arise in any tissue in which MLC-2a is expressed during prenatal or post-natal life. MLC-2a-related diseases and conditions can include diseases or conditions of the heart (e.g., heart failure, hypertrophy, or myopathy).

The invention provides several advantages. For example, using the diagnostic methods of the invention it is possible to detect an increased likelihood of diseases or conditions associated with MLC-2a, such as diseases of the heart, in a patient, so that appropriate intervention can be instituted before any symptoms occur. This may be useful, for example, with patients in high-risk groups for such diseases or conditions. Also, the diagnostic methods of the invention facilitate determination of the etiology of such an existing disease or condition in a patient, so that an appropriate approach to treatment can be selected. In addition, the screening methods of the invention can be used to identify compounds that can be used to treat or to prevent these diseases or conditions. The invention can also be used to treat diseases or conditions (e.g., heart failure) for which, prior to the invention, the only treatment was organ transplantation, which is limited by the availability of donor organs and the possibility of organ rejection.

Other features and advantages of the invention will be apparent from the following detailed description, the drawings, and the claims.

### BRIEF DESCRIPTION OF THE DRAWINGS

FIG. 1 is a schematic representation of the cDNA and amino acid sequences of zebrafish and human wild type Myosin Light Chain 2a. Also provided in FIG. 1 are the cDNA and amino acid sequences of the following zebrafish Myosin Light Chain 2 mutants: exon 3 missing, exons 3-5 missing, and intron 3 inserted.

FIG. 2 is an amino acid sequence alignment of zebrafish Myosin Light Chain 2a with xenopus, human, mouse, and drosophila orthologues.

FIG. 3 is schematic illustration of the effects of the splice donor site mutation in tel^{m225}.

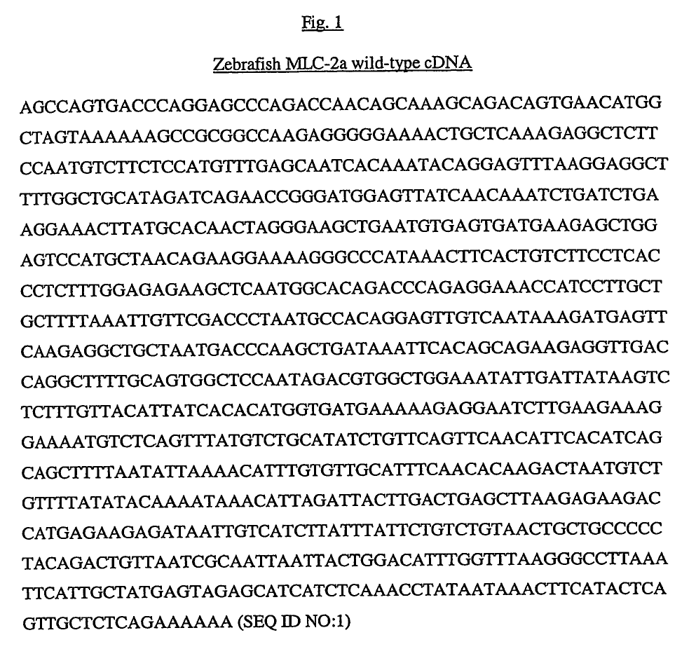

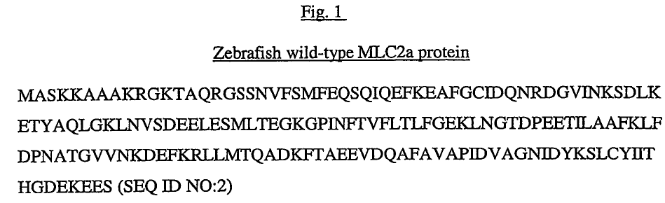

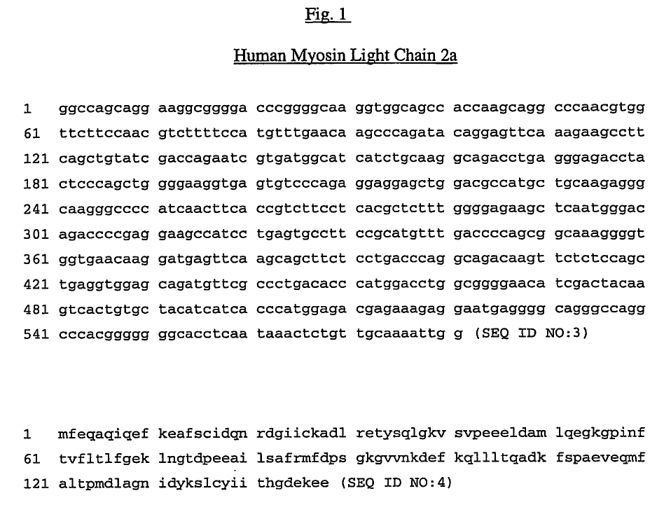

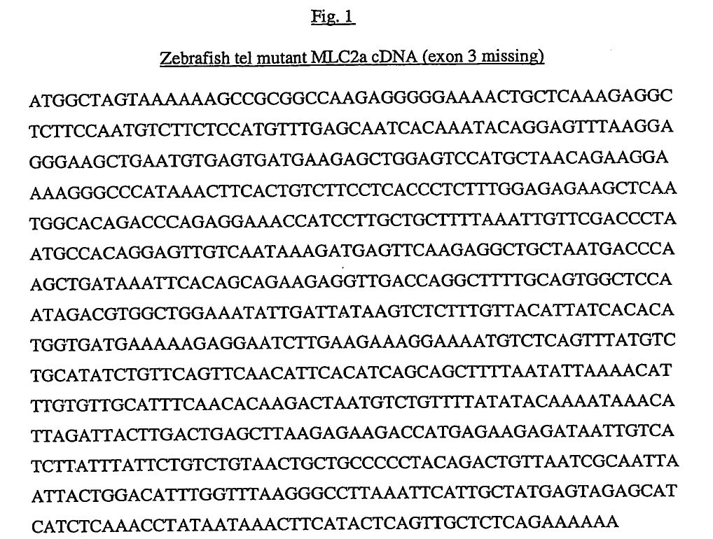

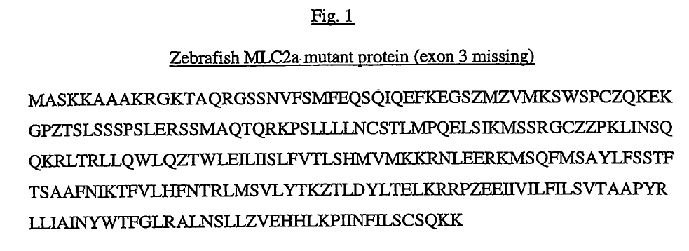

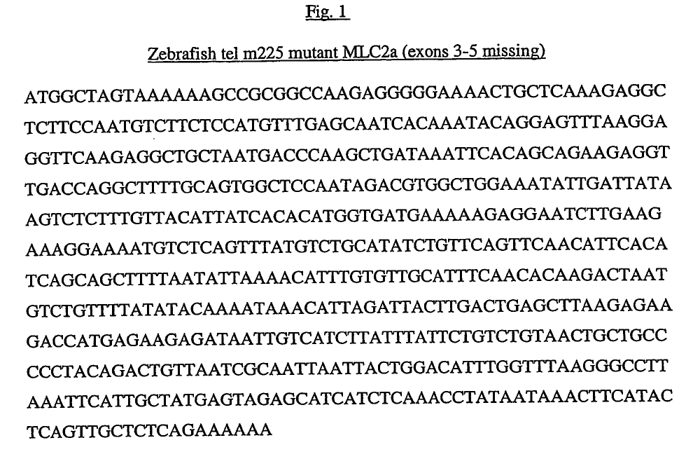

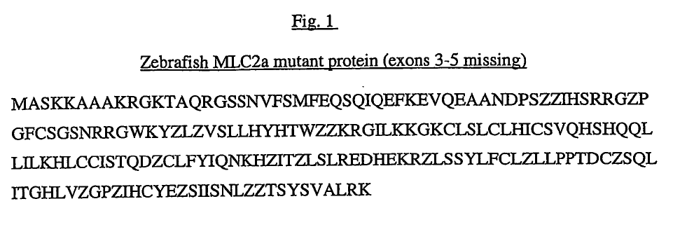

### DETAILED DESCRIPTION

The invention provides methods of diagnosing, preventing, and treating diseases and conditions associated with MLC-2a, such as diseases or conditions of the heart (also see below), and screening methods for identifying compounds that can be used to treat or prevent such diseases and conditions. In particular, we have identified a genetic mutation, tell tale heart (tel), which perturbs early embryonic heart contractility. We show here that the tel gene encodes Myosin Light Chain 2a (MLC-2a).

The diagnostic methods of the invention thus involve detection of mutations in genes encoding MLC-2a proteins, while the compound identification methods involve screening for compounds that affect the phenotype of organisms having mutations in genes encoding MLC-2a or other models of appropriate diseases and conditions. Compounds identified in this manner, as well as MLC-2a genes, proteins, and antibodies themselves, can be used in methods to treat or prevent diseases and conditions associated with MLC-2a.

The invention also provides animal model systems (e.g., zebrafish having mutations (e.g., the tell tale heart mutation) in MLC-2a genes, or mice (or other animals) having such mutations) that can be used in the screening methods mentioned above, as well as the MLC-2a protein, and genes encoding this protein. Also included in the invention are genes encoding mutant zebrafish MLC-2a proteins (e.g., genes having the tell tale heart mutation) and proteins encoded by these genes. Antibodies that specifically bind to these proteins (wild type or mutant) are also included in the invention.

The diagnostic, screening, and therapeutic methods of the invention, as well as the animal model systems, proteins, and genes of the invention, are described further, as follows, after a brief description of diseases and conditions associated with MLC-2a, which can be diagnosed, prevented, or treated according to the invention.

MLC-2a-Associated Diseases or Conditions

Abnormalities in MLC-2a genes or proteins can be associated with any of a wide variety of diseases or conditions, all of which can thus be diagnosed, prevented, or treated using the methods of the invention. For example, as discussed above, the tell tale heart mutation in zebrafish is characterized by abnormal heart contractility. Thus, detection of abnormalities in MLC-2a genes or their expression can be used in methods to diagnose, or to monitor the treatment or development of, diseases or conditions of heart (e.g., heart failure or cardiac hypertrophy). In addition, compounds that are identified in the screening methods described herein, as well as MLC-2a nucleic acid molecules, proteins, and antibodies themselves, can be used in methods to prevent or treat such diseases or conditions.

Examples of heart failure that can be diagnosed, prevented, or treated using the methods of the invention include congestive heart failure, which is characterized by fluid in the lungs or body, resulting from failure of the heart in acting as a pump; right sided heart failure (right ventricular), which is characterized by failure of the pumping action of the right ventricle, resulting in swelling of the body, especially the legs and abdomen; left sided heart failure (left ventricular), which is caused by failure of the pumping action of the left side of the heart, resulting in congestion of the lungs; forward heart failure, which is characterized by the inability of the heart to pump blood forward at a sufficient rate to meet the oxygen needs of the body at rest or during exercise; backward heart failure, which is characterized by the ability of the heart to meet the needs of the body only if heart filling pressures are abnormally high; low-output, which is characterized by failure to maintain blood output; and high-output, which is characterized by heart failure symptoms, even when cardiac output is high. Myosin Light Chain 2a also may play roles in cardiovascular diseases other than heart failure, such as coronary artery disease, heart fibrillation (e.g., atrial fibrillation), or conditions associated with valve formation defects, and, thus, detection of abnormalities in MLC2a genes or their expression can be used in methods to diagnose and monitor these conditions as well.

Diagnostic Methods

Nucleic acid molecules encoding MLC-2a proteins, as well as polypeptides encoded by these nucleic acid molecules and antibodies specific for these polypeptides, can be used in methods to diagnose or to monitor diseases and conditions involving mutations in, or inappropriate expression of, genes encoding this protein.

The diagnostic methods of the invention can be used, for example, with patients that have a disease or condition associated with MLC-2a, in an effort to determine its etiology and, thus, to facilitate selection of an appropriate course of treatment. The diagnostic methods can also be used with patients who have not yet developed, but who are at risk of developing, such a disease or condition, or with patients that are at an early stage of developing such a disease or condition. Also, the diagnostic methods of the invention can be used in prenatal genetic screening, for example, to identify parents who may be carriers of a recessive mutation in a gene encoding a MLC-2a protein. In addition, the methods can be used to investigate whether a MLC-2a mutation may be contributing to a disease or condition (e.g., heart disease) in a patient, by determining whether a MLC-2a gene of a patient includes a mutation. The methods of the invention can be used to diagnose (or to prevent or treat) the disorders described herein in any mammal, for example, in humans, domestic pets, or livestock.

Abnormalities in MLC-2a that can be detected using the diagnostic methods of the invention include those characterized by, for example, (i) a gene encoding a MLC-2a protein containing a mutation that results in the production of an abnormal MLC-2a protein, (ii) an abnormal MLC-2a polypeptide itself (e.g., a truncated protein), and (iii) a mutation in a MLC-2a gene that results in production of an abnormal amount of this protein. Detection of such abnormalities can be used to diagnose human diseases or conditions related to MLC-2a, such as those affecting the heart. Exemplary of the mutations in MLC-2a genes is the tell tale heart mutation, which is described further below.

A mutation in a MLC-2a gene can be detected in any tissue of a subject, even one in which this protein is not expressed. Because of the possibly limited number of tissues in which these proteins may be expressed, for limited time periods, and because of the possible undesirability of sampling such tissues (e.g., heart tissue) for assays, it may be preferable to detect mutant genes in other, more easily obtained sample types, such as in blood or amniotic fluid samples.

Detection of a mutation in a gene encoding a MLC-2a protein can be carried out using any standard diagnostic technique. For example, a biological sample obtained from a patient can be analyzed for one or more mutations (e.g., a tell tale heart mutation) in nucleic acid molecules encoding a MLC-2a protein using a mismatch detection approach. Generally, this approach involves polymerase chain reaction (PCR) amplification of nucleic acid molecules from a patient sample, followed by identification of a mutation (i.e., a mismatch) by detection of altered hybridization, aberrant electrophoretic gel migration, binding, or cleavage mediated by mismatch binding proteins, or by direct nucleic acid molecule sequencing. Any of these techniques can be used to facilitate detection of a mutant gene encoding a MLC-2a protein, and each is well known in the art. For instance, examples of these techniques are described by Orita et al. (Proc. Natl. Acad. Sci. U.S.A. 86:2766-2770, 1989) and Sheffield et al. (Proc. Natl. Acad. Sci. U.S.A. 86:232-236, 1989).

As noted above, in addition to facilitating diagnosis of an existing disease or condition, mutation detection assays also provide an opportunity to diagnose a predisposition to disease related to a mutation in a MLC-2a gene before the onset of symptoms. For example, a patient who is heterozygous for a gene encoding an abnormal MLC-2a protein (or an abnormal amount thereof) that suppresses normal MLC-2a biological activity or expression may show no clinical symptoms of a disease related to such proteins, and yet possess a higher than normal probability of developing such disease. Given such a diagnosis, a patient can take precautions to minimize exposure to adverse environmental factors, and can carefully monitor their medical condition, for example, through frequent physical examinations. As mentioned above, this type of diagnostic approach can also be used to detect a mutation in a gene encoding the MLC-2a protein in prenatal screens.

While it may be preferable to carry out diagnostic methods for detecting a mutation in a MLC-2a gene using genomic DNA from readily accessible tissues, as noted above, mRNA encoding this protein, or the protein itself, can also be assayed from tissue samples in which it is expressed. Expression levels of a gene encoding MLC-2a in such a tissue sample from a patient can be determined by using any of a number of standard techniques that are well known in the art, including northern blot analysis and quantitative PCR (see, e.g., Ausubel et al., supra; PCR Technology: Principles and Applications for DNA Amplification, H. A. Ehrlich, Ed., Stockton Press, NY; Yap et al. Nucl. Acids. Res. 19:4294, 1991).

In another diagnostic approach of the invention, an immunoassay is used to detect or to monitor the level of a MLC-2a protein in a biological sample. Polyclonal or monoclonal antibodies specific for the MLC-2a protein can be used in any standard immunoassay format (e.g., ELISA, Western blot, or RIA; see, e.g., Ausubel et al., supra) to measure polypeptide the levels of MLC-2a. These levels can be compared to levels of MLC-2a in a sample from an unaffected individual. Detection of a decrease in production of MLC-2a using this method, for example, may be indicative of a condition or a predisposition to a condition involving insufficient biological activity of the MLC-2a protein.

Immunohistochemical techniques can also be utilized for detection of MLC-2a protein in patient samples. For example, a tissue sample can be obtained from a patient, sectioned, and stained for the presence of MLC-2a using an anti-MLC-2a antibody and any standard detection system (e.g., one that includes a secondary antibody conjugated to an enzyme, such as horseradish peroxidase). General guidance regarding such techniques can be found in, e.g., Bancroft et al., Theory and Practice of Histological Techniques, Churchill Livingstone, 1982, and Ausubel et al., supra.

Identification of Molecules that can be Used to Treat or to Prevent Diseases or Conditions Associated with MLC-2a

Identification of a mutation in the gene encoding MLC-2a as resulting in a phenotype that results in abnormal heart contractility facilitates the identification of molecules (e.g., small organic or inorganic molecules, antibodies, peptides, or nucleic acid molecules) that can be used to treat or to prevent diseases or conditions associated with MLC-2a, as discussed above. The effects of candidate compounds on such diseases or conditions can be investigated using, for example, the zebrafish system. As is mentioned above, the zebrafish, Danio rerio, is a convenient organism to use in the genetic analysis of development. It has an accessible and transparent embryo, allowing direct observation of organ function from the earliest stages of development, has a short generation time, and is fecund. As discussed further below, zebrafish and other animals having a MLC-2a mutation, such as the tell tale heart mutation, which can be used in these methods, are also included in the invention.

In one example of the screening methods of the invention, a zebrafish having a mutation in a gene encoding the MLC-2a protein (e.g., a zebrafish having the tell tale heart mutation) is contacted with a candidate compound, and the effect of the compound on the development of abnormal heart contractility, or on the status of such an existing abnormality, is monitored relative to an untreated, identically mutant control.

After a compound has been shown to have a desired effect in the zebrafish system, it can be tested in other models of heart disease, for example, in mice or other animals having a mutation in a gene encoding MLC-2a. Alternatively, testing in such animal model systems can be carried out in the absence of zebrafish testing.

Cell culture-based assays can also be used in the identification of molecules that increase or decrease MLC-2a levels or biological activity. According to one approach, candidate molecules are added at varying concentrations to the culture medium of cells expressing MLC-2a mRNA. MLC-2a biological activity is then measured using standard techniques. The measurement of biological activity can include the measurement of MLC-2a protein and nucleic acid molecule levels.

In general, novel drugs for the prevention or treatment of diseases related to mutations in genes encoding MLC-2a can be identified from large libraries of natural products, synthetic (or semi-synthetic) extracts, and chemical libraries using methods that are well known in the art. Those skilled in the field of drug discovery and development will understand that the precise source of test extracts or compounds is not critical to the screening methods of the invention and that dereplication, or the elimination of replicates or repeats of materials already known for their therapeutic activities for MLC-2a, can be employed whenever possible.

Candidate compounds to be tested include purified (or substantially purified) molecules or one or more components of a mixture of compounds (e.g., an extract or supernatant obtained from cells; Ausubel et al., supra), and such compounds further include both naturally occurring or artificially derived chemicals and modifications of existing compounds. For example, candidate compounds can be polypeptides, synthesized organic or inorganic molecules, naturally occurring organic or inorganic molecules, nucleic acid molecules, and components thereof.

Numerous sources of naturally occurring candidate compounds are readily available to those skilled in the art. For example, naturally occurring compounds can be found in cell (including plant, fungal, prokaryotic, and animal) extracts, mammalian serum, growth medium in which mammalian cells have been cultured, protein expression libraries, or fermentation broths. In addition, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PharmaMar, U.S.A (Cambridge, Mass.). Furthermore, libraries of natural compounds can be produced, if desired, according to methods that are known in the art, e.g., by standard extraction and fractionation.

Artificially derived candidate compounds are also readily available to those skilled in the art. Numerous methods are available for generating random or directed synthesis (e.g., semi-synthesis or total synthesis) of any number of chemical compounds, including, for example, saccharide-, lipid-, peptide-, and nucleic acid molecule-based compounds. In addition, synthetic compound libraries are commercially available from Brandon Associates (Merrimack, N.H.) and Aldrich Chemicals (Milwaukee, Wis.). Libraries of synthetic compounds can also be produced, if desired, according to methods known in the art, e.g., by standard extraction and fractionation. Furthermore, if desired, any library or compound can be readily modified using standard chemical, physical, or biochemical methods. The techniques of modern synthetic chemistry, including combinatorial chemistry, can also be used (reviewed in Schreiber, Bioorganic and Medicinal Chemistry 6:1172-1152, 1998; Schreiber, Science 287:1964-1969, 2000).

When a crude extract is found to have an effect on the development or persistence of a MLC-2a-associated disease, further fractionation of the positive lead extract can be carried out to isolate chemical constituents responsible for the observed effect. Thus, the goal of the extraction, fractionation, and purification process is the careful characterization and identification of a chemical entity within the crude extract having a desired activity. The same assays described herein for the detection of activities in mixtures of compounds can be used to purify the active component and to test derivatives of these compounds. Methods of fractionation and purification of such heterogeneous extracts are well known in the art. If desired, compounds shown to be useful agents for treatment can be chemically modified according to methods known in the art.

In general, compounds that are found to activate MLC-2a expression or activity may be used in the prevention or treatment of diseases or conditions of heart, such as those that are characterized by abnormal growth or development, or heart failure (also see above).

Animal Model Systems

The invention also provides animal model systems for use in carrying out the screening methods described above. Examples of these model systems include zebrafish and other animals, such as mice, that have a mutation (e.g., the tell tale heart mutation) in a MLC-2a gene. For example, a zebrafish model that can be used in the invention can include a mutation that results in a lack of MLC-2a protein production or production of a truncated (e.g., by introduction of a stop codon or a splice site mutation) or otherwise altered MLC-2a gene product. As a specific example, a zebrafish having the tell tale heart mutation can be used (see below).

Treatment or Prevention of MLC-2a-Associated Diseases or Conditions

Compounds identified using the screening methods described above can be used to treat patients that have or are at risk of developing diseases or conditions of the heart (e.g., heart failure or cardiac hypertrophy). Nucleic acid molecules encoding the MLC-2a protein, as well as these proteins themselves, can also be used in such methods. Treatment may be required only for a short period of time or may, in some form, be required throughout a patient's lifetime. Any continued need for treatment, however, can be determined using, for example, the diagnostic methods described above. In considering various therapies, it is to be understood that such therapies are, preferably, targeted to the affected or potentially affected organ (e.g., the heart). Such targeting can be achieved using standard methods.

Treatment or prevention of diseases resulting from a mutated MLC-2a gene can be accomplished, for example, by modulating the function of a mutant MLC-2a protein. Treatment can also be accomplished by delivering normal MLC-2a protein to appropriate cells, altering the levels of normal or mutant MLC-2a protein, replacing a mutant gene encoding a MLC-2a protein with a normal gene encoding a MLC-2a protein, or administering a normal gene encoding a MLC-2a protein. It is also possible to correct the effects of a defect in a gene encoding a MLC-2a protein by modifying the physiological pathway (e.g., a signal transduction pathway) in which a MLC-2a protein participates.

In a patient diagnosed as being heterozygous for a gene encoding a mutant MLC-2a protein, or as susceptible to such mutations or aberrant MLC-2a expression (even if those mutations or expression patterns do not yet result in alterations in expression or biological activity of MLC-2a), any of the therapies described herein can be administered before the occurrence of the disease phenotype. In particular, compounds shown to have an effect on the phenotype of mutants, or to modulate expression of MLC-2a proteins, can be administered to patients diagnosed with potential or actual disease by any standard dosage and route of administration.

Any appropriate route of administration can be employed to administer a compound identified as described above, a MLC-2a gene, protein, or antibody, according to the invention. For example, administration can be parenteral, intravenous, intra-arterial, subcutaneous, intramuscular, intraventricular, intracapsular, intraspinal, intracistemal, intraperitoneal, intranasal, by aerosol, by suppository, or oral.

A therapeutic compound of the invention can be administered within a pharmaceutically acceptable diluent, carrier, or excipient, in unit dosage form. Administration can begin before or after the patient is symptomatic. Methods that are well known in the art for making formulations are found, for example, in Remington's Pharmaceutical Sciences (18^{th} edition), ed. A. Gennaro, 1990, Mack Publishing Company, Easton, Pa. Therapeutic formulations can be in the form of liquid solutions or suspensions. Formulations for parenteral administration can contain, for example, excipients, sterile water, or saline; polyalkylene glycols, such as polyethylene glycol; oils of vegetable origin; or hydrogenated napthalenes. Biocompatible, biodegradable lactide polymer, lactide/glycolide copolymer, or polyoxyethylene-polyoxypropylene copolymers can be used to control the release of the compounds. Other potentially useful parenteral delivery systems include ethylene-vinyl acetate copolymer particles, osmotic pumps, implantable infusion systems, and liposomes. For oral administration, formulations can be in the form of tablets or capsules. Formulations for inhalation can contain excipients, for example, lactose, or can be aqueous solutions containing, for example, polyoxyethylene-9-lauryl ether, glycocholate, and deoxycholate, or can be oily solutions for administration in the form of nasal drops or as a gel. Alternatively, intranasal formulations can be in the form of powders or aerosols.

To replace a mutant protein with normal protein, or to add protein to cells that do not express a sufficient amount of MLC-2a or normal MLC-2a, it may be necessary to obtain large amounts of pure MLC-2a protein from cell culture systems in which the protein is expressed (see, e.g., below). Delivery of the protein to the affected tissue can then be accomplished using appropriate packaging or administration systems.

Gene therapy is another therapeutic approach for preventing or ameliorating diseases caused by MLC-2a gene defects. Nucleic acid molecules encoding wild type MLC-2a protein can be delivered to cells that lack sufficient, normal MLC-2a protein biological activity (e.g., cells carrying mutations (e.g., the tell tale heart mutation) in MLC-2a genes). The nucleic acid molecules must be delivered to those cells in a form in which they can be taken up by the cells and so that sufficient levels of protein, to provide effective MLC-2a protein function, can be produced. Alternatively, for some MLC-2a mutations, it may be possible to slow the progression of the resulting disease or to modulate MLC-2a protein activity by introducing another copy of a homologous gene bearing a second mutation in that gene, to alter the mutation, or to use another gene to block any negative effect.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, the full length MLC-2a gene, or a portion thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cometta et al., Nucleic Acid Research and Molecular Biology 36:311-322, 1987; Anderson, Science 226:401-409, 1984; Moen, Blood Cells 17:407416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the introduction of therapeutic DNA into cells predicted to be subject to diseases involving the MLC-2a protein. For example, a MLC-2a nucleic acid molecule or an antisense nucleic acid molecule can be introduced into a cell by lipofection (Felgner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990).

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal MLC-2a protein into a cultivatable cell type ex vivo (e.g., an autologous or heterologous primary cell or progeny thereof), after which the cell (or its descendants) are injected into a targeted tissue.

MLC-2a cDNA expression for use in gene therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct MLC-2a expression. The enhancers used can include, without limitation, those that are characterized as tissue- or cell-specific enhancers. Alternatively, if a MLC-2a genomic clone is used as a therapeutic construct (such clones can be identified by hybridization with MLC-2a cDNA, as described herein), regulation can be mediated by the cognate regulatory sequences or, if desired, by regulatory sequences derived from a heterologous source, including any of the promoters or regulatory elements described above.

Molecules for effecting antisense-based strategies can be employed to explore MLC-2a protein gene function, as a basis for therapeutic drug design, as well as to treat MLC-2a-associated diseases. These strategies are based on the principle that sequence-specific suppression of gene expression (via transcription or translation) can be achieved by intracellular hybridization between genomic DNA or mRNA and a complementary antisense species. The formation of a hybrid RNA duplex interferes with transcription of the target MLC-2a-encoding genomic DNA molecule, or processing, transport, translation, or stability of the target MLC-2a mRNA molecule.

Antisense strategies can be delivered by a variety of approaches. For example, antisense oligonucleotides or antisense RNA can be directly administered (e.g., by intravenous injection) to a subject in a form that allows uptake into cells. Alternatively, viral or plasmid vectors that encode antisense RNA (or antisense RNA fragments) can be introduced into a cell in vivo or ex vivo. Antisense effects can be induced by control (sense) sequences; however, the extent of phenotypic changes is highly variable. Phenotypic effects induced by antisense molecules are based on changes in criteria such as protein levels, protein activity measurement, and target mRNA levels.

MLC-2a gene therapy can also be accomplished by direct administration of antisense MLC-2a mRNA to a cell that is expected to be adversely affected by the expression of wild type or mutant MLC-2a protein. The antisense MLC-2a mRNA can be produced and isolated by any standard technique, but is most readily produced by in vitro transcription using an antisense MLC-2a cDNA under the control of a high efficiency promoter (e.g., the T7 promoter). Administration of antisense MLC-2a mRNA to cells can be carried out by any of the methods for direct nucleic acid molecule administration described above.

An alternative strategy for inhibiting MLC-2a protein function using gene therapy involves intracellular expression of an anti-MLC-2a protein antibody or a portion of an anti-MLC-2a protein antibody. For example, the gene (or gene fragment) encoding a monoclonal antibody that specifically binds to a MLC-2a protein and inhibits its biological activity can be placed under the transcriptional control of a tissue-specific gene regulatory sequence.

Another therapeutic approach included in the invention involves administration of a recombinant MLC-2a polypeptide, either directly to the site of a potential or actual disease-affected tissue (for example, by injection) or systemically (for example, by any conventional recombinant protein administration technique). The dosage of the MLC-2a protein depends on a number of factors, including the size and health of the individual patient but, generally, between 0.1 mg and 100 mg, inclusive, is administered per day to an adult in any pharmaceutically acceptable formulation.

In addition to the therapeutic methods described herein, involving administration of MLC-2a-modulating compounds, MLC-2a proteins, or MLC-2a nucleic acids to patients, the invention provides methods of culturing organs in the presence of such molecules. In particular, as is noted above, a MLC-2a mutation is associated with abnormal heart contractility. Thus, culturing heart tissue in the presence of these molecules can be used to promote its proper contractility. This tissue can be that which is being prepared for transplant from, e.g., an allogeneic or xenogeneic donor, as well as synthetic tissue or organs.

Synthesis of MLC-2a Proteins, Polypeptides, and Polypeptide Fragments

Those skilled in the art of molecular biology will understand that a wide variety of expression systems can be used to produce recombinant MLC-2a proteins. As discussed further below, the precise host cell used is not critical to the invention. The MLC-2a proteins can be produced in a prokaryotic host (e.g., E. coli) or in a eukaryotic host (e.g., S. cerevisiae, insect cells, such as Sf9 cells, or mammalian cells, such as COS-1, NIH 3T3, or HeLa cells). These cells are commercially available from, for example, the American Type Culture Collection, Manassas, Va. (see also Ausubel et al., supra). The method of transformation and the choice of expression vehicle (e.g., expression vector) will depend on the host system selected. Transformation and transfection methods are described, e.g., in Ausubel et al., supra, and expression vehicles can be chosen from those provided, e.g., in Pouwels et al., Cloning Vectors: A Laboratory Manual, 1985, Supp. 1987. Specific examples of expression systems that can be used in the invention are described further as follows.

For protein expression, eukaryotic or prokaryotic expression systems can be generated in which MLC-2a gene sequences are introduced into a plasmid or other vector, which is then used to transform living cells. Constructs in which full-length MLC-2a cDNAs, containing the entire open reading frame, inserted in the correct orientation into an expression plasmid, can be used for protein expression. Alternatively, portions of MLC-2a gene sequences, including wild type or mutant MLC-2a sequences, can be inserted. Prokaryotic and eukaryotic expression systems allow various important functional domains of MLC-2a proteins to be recovered, if desired, as fusion proteins, and then used for binding, structural, and functional studies, and also for the generation of antibodies.

Typical expression vectors contain promoters that direct synthesis of large amounts of mRNA corresponding to a nucleic acid molecule that has been inserted into the vector. They can also include a eukaryotic or prokaryotic origin of replication, allowing for autonomous replication within a host cell, sequences that confer resistance to an otherwise toxic drug, thus allowing vector-containing cells to be selected in the presence of the drug, and sequences that increase the efficiency with which the synthesized mRNA is translated. Stable, long-term vectors can be maintained as freely replicating entities by using regulatory elements of, for example, viruses (e.g., the OriP sequences from the Epstein Barr Virus genome). Cell lines can also be produced that have the vector integrated into genomic DNA of the cells and, in this manner, the gene product can be produced in the cells on a continuous basis.

Expression of foreign molecules in bacteria, such as Escherichia coli, requires the insertion of a foreign nucleic acid molecule, e.g., a MLC-2a nucleic acid molecule, into a bacterial expression vector. Such plasmid vectors include several elements required for the propagation of the plasmid in bacteria, and for expression of foreign DNA contained within the plasmid. Propagation of only plasmid-bearing bacteria is achieved by introducing, into the plasmid, a selectable marker-encoding gene that allows plasmid-bearing bacteria to grow in the presence of an otherwise toxic drug. The plasmid also contains a transcriptional promoter capable of directing synthesis of large amounts of mRNA from the foreign DNA. Such promoters can be, but are not necessarily, inducible promoters that initiate transcription upon induction by culture under appropriate conditions (e.g., in the presence of a drug that activates the promoter). The plasmid also, preferably, contains a polylinker to simplify insertion of the gene in the correct orientation within the vector.

Once an appropriate expression vector containing a MLC-2a gene, or a fragment, fusion, or mutant thereof, is constructed, it can be introduced into an appropriate host cell using a transformation technique, such as, for example, calcium phosphate transfection, DEAE-dextran transfection, electroporation, microinjection, protoplast fusion, or liposome-mediated transfection. Host cells that can be transfected with the vectors of the invention can include, but are not limited to, E. coli or other bacteria, yeast, fungi, insect cells (using, for example, baculoviral vectors for expression), or cells derived from mice, humans, or other animals. Mammalian cells can also be used to express MLC-2a proteins using a virus expression system (e.g., a vaccinia virus expression system) described, for example, in Ausubel et al., supra.

In vitro expression of MLC-2a proteins, fusions, polypeptide fragments, or mutants encoded by cloned DNA can also be carried out using the T7 late-promoter expression system. This system depends on the regulated expression of T7 RNA polymerase, an enzyme encoded in the DNA of bacteriophage T7. The T7 RNA, polymerase initiates transcription at a specific 23 base pair promoter sequence called the T7 late promoter. Copies of the T7 late promoter are located at several sites on the T7 genome, but none are present in E. coli chromosomal DNA. As a result, in T7-infected E. coli, T7 RNA polymerase catalyzes transcription of viral genes, but not E. coli genes. In this expression system, recombinant E. coli cells are first engineered to carry the gene encoding T7 RNA polymerase next to the lac promoter. In the presence of IPTG, these cells transcribe the T7 polymerase gene at a high rate and synthesize abundant amounts of T7 RNA polymerase. These cells are then transformed with plasmid vectors that carry a copy of the T7 late promoter protein. When IPTG is added to the culture medium containing these transformed E. coli cells, large amounts of T7 RNA polymerase are produced. The polymerase then binds to the T7 late promoter on the plasmid expression vectors, catalyzing transcription of the inserted cDNA at a high rate. Since each E. coli cell contains many copies of the expression vector, large amounts of mRNA corresponding to the cloned cDNA can be produced in this system and the resulting protein can be radioactively labeled.

Plasmid vectors containing late promoters and the corresponding RNA polymerases from related bacteriophages, such as T3, T5, and SP6, can also be used for in vitro production of proteins from cloned DNA. E. coli can also be used for expression using an M13 phage, such as mGPI-2. Furthermore, vectors that contain phage lambda regulatory sequences, or vectors that direct the expression of fusion proteins, for example, a maltose-binding protein fusion protein or a glutathione-S-transferase fusion protein, also can be used for expression in E. coli.

Eukaryotic expression systems are useful for obtaining appropriate post-translational modification of expressed proteins. Transient transfection of a eukaryotic expression plasmid containing a MLC-2a gene into a eukaryotic host cell allows the transient production of a MLC-2a protein by the transfected host cell. MLC-2a proteins can also be produced by a stably-transfected eukaryotic (e.g., mammalian) cell line. A number of vectors suitable for stable transfection of mammalian cells are available to the public (see, e.g., Pouwels et al., supra), as are methods for constructing lines including such cells (see, e.g., Ausubel et al., supra).

In one example, cDNA encoding a MLC-2a protein, fusion, mutant, or polypeptide fragment is cloned into an expression vector that includes the dihydrofolate reductase (DHFR) gene. Integration of the plasmid and, therefore, integration of the tell tale heart protein-encoding gene, into the host cell chromosome is selected for by inclusion of 0.01-300 μM methotrexate in the cell culture medium (Ausubel et al., supra). This dominant selection can be accomplished in most cell types. Recombinant protein expression can be increased by DHFR-mediated amplification of the transfected gene. Methods for selecting cell lines bearing gene amplifications are described in Ausubel et al., supra. These methods generally involve extended culture in medium containing gradually increasing levels of methotrexate. The most commonly used DHFR-containing expression vectors are PCVSEII-DHFR and pAdD26SV(A) (described, for example, in Ausubel et al., supra). The host cells described above or, preferably, a DHFR-deficient CHO cell line (e.g., CHO DHFR-cells, ATCC Accession No. CRL 9096) are among those that are most preferred for DHFR selection of a stably transfected cell line or DHFR-mediated gene amplification.

Another preferred eukaryotic expression system is the baculovirus system using, for example, the vector pBacPAK9, which is available from Clontech (Palo Alto, Calif.). If desired, this system can be used in conjunction with other protein expression techniques, for example, the myc tag approach described by Evan et al. (Molecular and Cellular Biology 5:3610-3616, 1985).

Once a recombinant protein is expressed, it can be isolated from the expressing cells by cell lysis followed by protein purification techniques, such as affinity chromatography. In this example, an anti-MLC-2a antibody, which can be produced by the methods described herein, can be attached to a column and used to isolate the recombinant MLC-2a. Lysis and fractionation of MLC-2a-harboring cells prior to affinity chromatography can be performed by standard methods (see, e.g., Ausubel et al., supra). Once isolated, the recombinant protein can, if desired, be purified further by, e.g., high performance liquid chromatography (HPLC; e.g., see Fisher, Laboratory Techniques In Biochemistry and Molecular Biology, Work and Burdon, Eds., Elsevier, 1980).

Polypeptides of the invention, particularly short MLC-2a fragments and longer fragments of the N-terminus and C-terminus of MLC-2a, can also be produced by chemical synthesis (e.g., by the methods described in Solid Phase Peptide Synthesis, 2^{nd} ed., 1984, The Pierce Chemical Co., Rockford, Ill.). These general techniques of polypeptide expression and purification can also be used to produce and isolate useful MLC-2a fragments or analogs, as described herein.

MLC-2a Protein Fragments

Polypeptide fragments that include various portions of MLC-2a proteins are useful in identifying the domains of MLC-2a that are important for its biological activities. Methods for generating such fragments are well known in the art (see, for example, Ausubel et al., supra), using the nucleotide sequences provided herein. For example, a MLC-2a protein fragment can be generated by PCR amplifying a desired MLC-2a nucleic acid molecule fragment using oligonucleotide primers designed based upon MLC-2a nucleic acid sequences. Preferably, the oligonucleotide primers include unique restriction enzyme sites that facilitate insertion of the amplified fragment into the cloning site of an expression vector (e.g., a mammalian expression vector, see above). This vector can then be introduced into a cell (e.g., a mammalian cell; see above) by artifice, using any of the various techniques that are known in the art, such as those described herein, resulting in the production of a MLC-2a protein fragment in the cell containing the expression vector. MLC-2a protein fragments (e.g., chimeric fusion proteins) can also be used to raise antibodies specific for various regions of the MLC-2a protein using, for example, the methods described below. In some instances, it maybe desirable to include conserved domains (e.g., calcium binding domains) in fragments, while in others, less conserved domains can be included.

MLC-2a Protein Antibodies

To prepare polyclonal antibodies, MLC-2a proteins, fragments of MLC-2a proteins, or fusion proteins containing defined portions of MLC-2a proteins can be synthesized in, e.g., bacteria by expression of corresponding DNA sequences contained in a suitable cloning vehicle. Fusion proteins are commonly used as a source of antigen for producing antibodies. Two widely used expression systems for E. coli are lacZ fusions using the pUR series of vectors and trpE fusions using the pATH vectors. The proteins can be purified, coupled to a carrier protein, mixed with Freund's adjuvant to enhance stimulation of the antigenic response in an inoculated animal, and injected into rabbits or other laboratory animals. Alternatively, protein can be isolated from MLC-2a-expressing cultured cells. Following booster injections at bi-weekly intervals, the rabbits or other laboratory animals are then bled and the sera isolated. The sera can be used directly or can be purified prior to use by various methods, including affinity chromatography employing reagents such as Protein A-Sepharose, antigen-Sepharose, and anti-mouse-Ig-Sepharose. The sera can then be used to probe protein extracts from MLC-2a-expressing tissue fractionated by polyacrylamide gel electrophoresis to identify MLC-2a proteins. Alternatively, synthetic peptides can be made that correspond to antigenic portions of the protein and used to inoculate the animals.

To generate peptide or full-length protein for use in making, for example, MLC-2a-specific antibodies, a MLC-2a coding sequence can be expressed as a C— terminal or N-terminal fusion with glutathione S-transferase (GST; Smith et al., Gene 67:31-40, 1988). The fusion protein can be purified on glutathione-Sepharose beads, eluted with glutathione, cleaved with a protease, such as thrombin or Factor-Xa (at the engineered cleavage site), and purified to the degree required to successfully immunize rabbits. Primary immunizations can be carried out with Freund's complete adjuvant and subsequent immunizations performed with Freund's incomplete adjuvant. Antibody titers can be monitored by Western blot and immunoprecipitation analyses using the protease-cleaved MLC-2a fragment of the GST-MLC-2a protein. immune sera can be affinity purified using CNBr-Sepharose-coupled MLC-2a. Antiserum specificity can be determined using a panel of unrelated GST fusion proteins.

Alternatively, monoclonal MLC-2a antibodies can be produced by using, as an antigen, MLC-2a isolated from MLC-2a-expressing cultured cells or MLC-2a protein isolated from tissues. The cell extracts, or recombinant protein extracts containing MLC-2a, can, for example, be injected with Freund's adjuvant into mice. Several days after being injected, the mouse spleens can be removed, the tissues disaggregated, and the spleen cells suspended in phosphate buffered saline (PBS). The spleen cells serve as a source of lymphocytes, some of which would be producing antibody of the appropriate specificity. These can then be fused with permanently growing myeloma partner cells, and the products of the fusion plated into a number of tissue culture wells in the presence of selective agents, such as hypoxanthine, aminopterine, and thymidine (HAT). The wells can then be screened by ELISA to identify those containing cells making antibodies capable of binding to MLC-2a, polypeptide fragment, or mutant thereof. These cells can then be re-plated and, after a period of growth, the wells containing these cells can be screened again to identify antibody-producing cells. Several cloning procedures can be carried out until over 90% of the wells contain single clones that are positive for specific antibody production. From this procedure, a stable line of clones that produce the antibody can be established. The monoclonal antibody can then be purified by affinity chromatography using Protein A Sepharose and ion exchange chromatography, as well as variations and combinations of these techniques. Once produced, monoclonal antibodies are also tested for specific MLC-2a recognition by Western blot or immunoprecipitation analysis (see, e.g., Kohler et al., Nature 256:495, 1975; Kohler et al., European Journal of Immunology 6:511, 1976; Kohler et al., European Journal of Immunology 6:292, 1976; Hammerling et al., In Monoclonal Antibodies and T Cell Hybridomas, Elsevier, New York, N.Y., 1981; Ausubel et al., supra).

As an alternate or adjunct immunogen to GST fusion proteins, peptides corresponding to relatively unique hydrophilic regions of MLC-2a can be generated and coupled to keyhole limpet hemocyanin (KLH) through an introduced C-terminal lysine. Antiserum to each of these peptides can be similarly affinity-purified on peptides conjugated to BSA, and specificity tested by ELISA and Western blotting using peptide conjugates, and by Western blotting and immunoprecipitation using MLC-2a, for example, expressed as a GST fusion protein.

Antibodies of the invention can be produced using MLC-2a amino acid sequences that do not reside within highly conserved regions, and that appear likely to be antigenic, as analyzed by criteria such as those provided by the Peptide Structure Program (Genetics Computer Group Sequence Analysis Package, Program Manual for the GCG Package, Version 7, 1991) using the algorithm of Jameson et al., CABIOS 4:181, 1988. These fragments can be generated by standard techniques, e.g., by PCR, and cloned into the pGEX expression vector. GST fusion proteins can be expressed in E. coli and purified using a glutathione-agarose affinity matrix (Ausubel et al., supra). To generate rabbit polyclonal antibodies, and to minimize the potential for obtaining antisera that is non-specific, or exhibits low-affinity binding to MLC-2a, two or three fusions are generated for each protein, and each fusion is injected into at least two rabbits. Antisera are raised by injections in series, preferably including at least three booster injections.

In addition to intact monoclonal and polyclonal anti-MLC-2a antibodies, the invention features various genetically engineered antibodies, humanized antibodies, and antibody fragments, including F(ab′)2, Fab′, Fab, Fv, and sFv fragments. Truncated versions of monoclonal antibodies, for example, can be produced by recombinant methods in which plasmids are generated that express the desired monoclonal antibody fragment(s) in a suitable host. Antibodies can be humanized by methods known in the art, e.g., monoclonal antibodies with a desired binding specificity can be commercially humanized (Scotgene, Scotland; Oxford Molecular, Palo Alto, Calif.). Fully human antibodies, such as those expressed in transgenic animals, are also included in the invention (Green et al., Nature Genetics 7:13-21, 1994).

Ladner (U.S. Pat. Nos. 4,946,778 and 4,704,692) describes methods for preparing single polypeptide chain antibodies. Ward et al., Nature 341:544-546, 1989, describes the preparation of heavy chain variable domains, which they term “single domain antibodies,” and which have high antigen-binding affinities. McCafferty et al., Nature 348:552-554, 1990, shows that complete antibody V domains can be displayed on the surface of fd bacteriophage, that the phage bind specifically to antigen, and that rare phage (one in a million) can be isolated after affinity chromatography. Boss et al., U.S. Pat. No. 4,816,397, describes various methods for producing immunoglobulins, and immunologically functional fragments thereof, that include at least the variable domains of the heavy and light chains in a single host cell. Cabilly et al., U.S. Pat. No. 4,816,567, describes methods for preparing chimeric antibodies.

Use of MLC-2a Antibodies

Antibodies to MLC-2a can be used, as noted above, to detect MLC-2a or to inhibit the biological activities of MLC-2a. For example, a nucleic acid molecule encoding an antibody or portion of an antibody can be expressed within a cell to inhibit MLC-2a function. In addition, the antibodies can be coupled to compounds, such as radionuclides and liposomes, for diagnostic or therapeutic uses. Antibodies that inhibit the activity of a MLC-2a polypeptide described herein can also be useful in preventing or slowing the development of a disease caused by inappropriate expression of a wild type or mutant MLC-2a gene.

Detection of MLC-2a Gene Expression

As noted, the antibodies described above can be used to monitor MLC-2a gene expression. In situ hybridization of RNA can be used to detect the expression of MLC-2a genes. RNA in situ hybridization techniques rely upon the hybridization of a specifically labeled nucleic acid probe to the cellular RNA in individual cells or tissues. Therefore, RNA in situ hybridization is a powerful approach for studying tissue- and temporal-specific gene expression. In this method, oligonucleotides, cloned DNA fragments, or antisense RNA transcripts of cloned DNA fragments corresponding to unique portions of MLC-2a genes are used to detect specific mRNA species, e.g., in the tissues of animals, such as mice, at various developmental stages. Other gene expression detection techniques are known to those of skill in the art and can be employed for detection of MLC-2a gene expression.

Identification of Additional MLC-2a Genes

Standard techniques, such as the polymerase chain reaction (PCR) and DNA hybridization, can be used to clone MLC-2a gene homologues in other species and MLC-2a-related genes in humans. MLC-2a-related genes and homologues can be readily identified using low-stringency DNA hybridization or low-stringency PCR with human MLC-2a probes or primers. Degenerate primers encoding human MLC-2a or human MLC-2a-related amino acid sequences can be used to clone additional MLC-2a-related genes and homologues by RT-PCR.

Construction of Transgenic Animals and Knockout Animals

Characterization of MLC-2a genes provides information that allows MLC-2a knockout animal models to be developed by homologous recombination. Preferably, a MLC-2a knockout animal is a mammal, most preferably a mouse. Similarly, animal models of MLC-2a overproduction can be generated by integrating one or more MLC-2a sequences into the genome of an animal, according to standard transgenic techniques. Moreover, the effect of MLC-2a mutations (e.g., dominant gene mutations) can be studied using transgenic mice carrying mutated MLC-2a transgenes or by introducing such mutations into the endogenous MLC-2a gene, using standard homologous recombination techniques.

A replacement-type targeting vector, which can be used to create a knockout model, can be constructed using an isogenic genomic clone, for example, from a mouse strain such as 129/Sv (Stratagene Inc., LaJolla, Calif.). The targeting vector can be introduced into a suitably derived line of embryonic stem (ES) cells by electroporation to generate ES cell lines that carry a profoundly truncated form of a MLC-2a gene. To generate chimeric founder mice, the targeted cell lines are injected into a mouse blastula-stage embryo. Heterozygous offspring can be interbred to homozygosity. MLC-2a knockout mice provide a tool for studying the role of MLC-2a in embryonic development and in disease. Moreover, such mice provide the means, in vivo, for testing therapeutic compounds for amelioration of diseases or conditions involving MLC-2a-dependent or a MLC-2a-effected pathway.

Use of MLC-2a as a Marker for Stem Cells of the Heart

As MLC-2a is expressed in cells that give rise to the heart during the course of development, it can be used as a marker for stem cells of the heart. For example, MLC-2a can be used to identify, sort, or target such stem cells. A pool of candidate cells, for example, can be analyzed for MLC-2a expression, to facilitate the identification of heart stem cells, which, based on this identification can be separated from the pool. The isolated stem cells can be used for many purposes that are known to those of skill in this art. For example, the stem cells can be used in the production of new organs, in organ culture, or to fortify damaged or transplanted organs.

Experimental Results

tell tale heart (tel^{m225}) is an ENU-induced recessive embryonic lethal mutation in zebrafish that selectively perturbs early embryonic heart contractility. tel mutant embryos form an apparently morphologically normal heart tube, containing both endocardial and myocardial layers, in anatomically correct positions. The first evident defect in these mutants is a dramatically reduction in peristaltic contraction waves traversing the heart tube. tel mutants develop two cardiac chambers, which loop and display normal cell number and size at 72 hours post fertilization. However, cardiac contractility is severely impaired and only in the atrio-ventricular canal, rhythmic contractions can be observed. While myocardial cells of the ventricle do not contract at all, weak regional contractions can be observed in the atrial chamber. Blood circulation is never established and tel mutant embryos die on day 7 post fertilization.

Bulked segregant analysis assigned tel to zebrafish linkage group 8. Meiotic fine mapping placed the tel locus in a 1.4 cM interval between microsatellite markers Z25210. Further testing of recombination events for ESTs mapping to the interval revealed only one recombination in 4498 meiotic events for EST clone fb57f10.

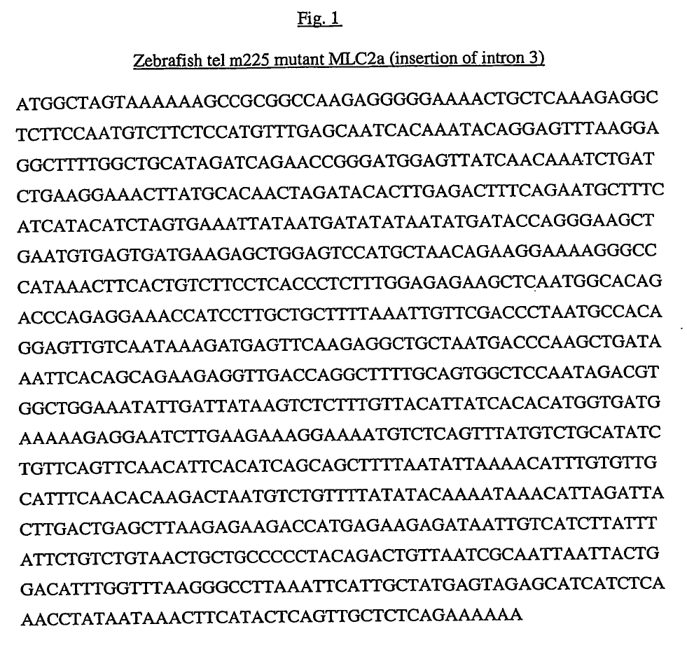

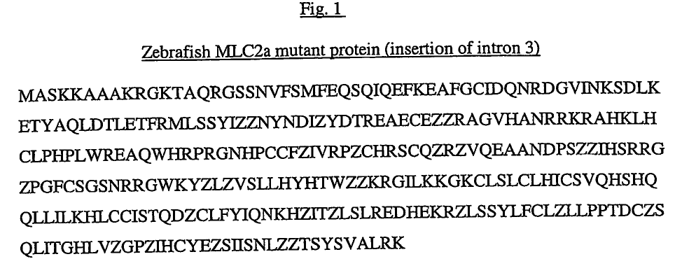

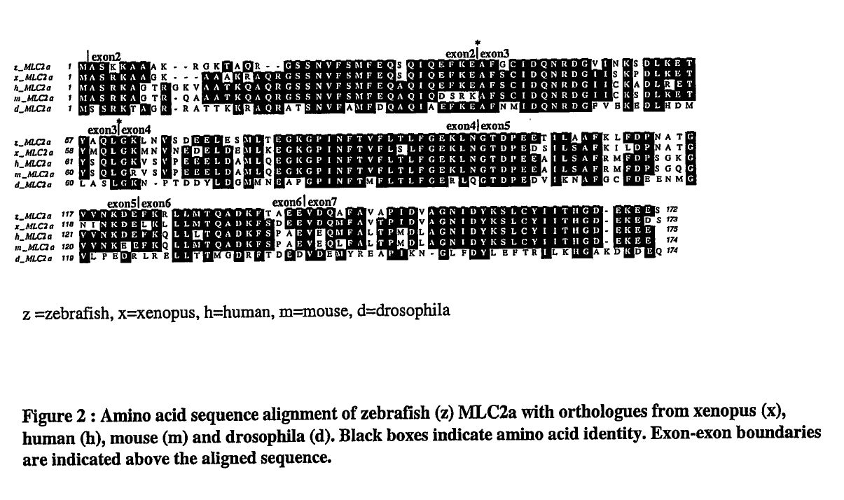

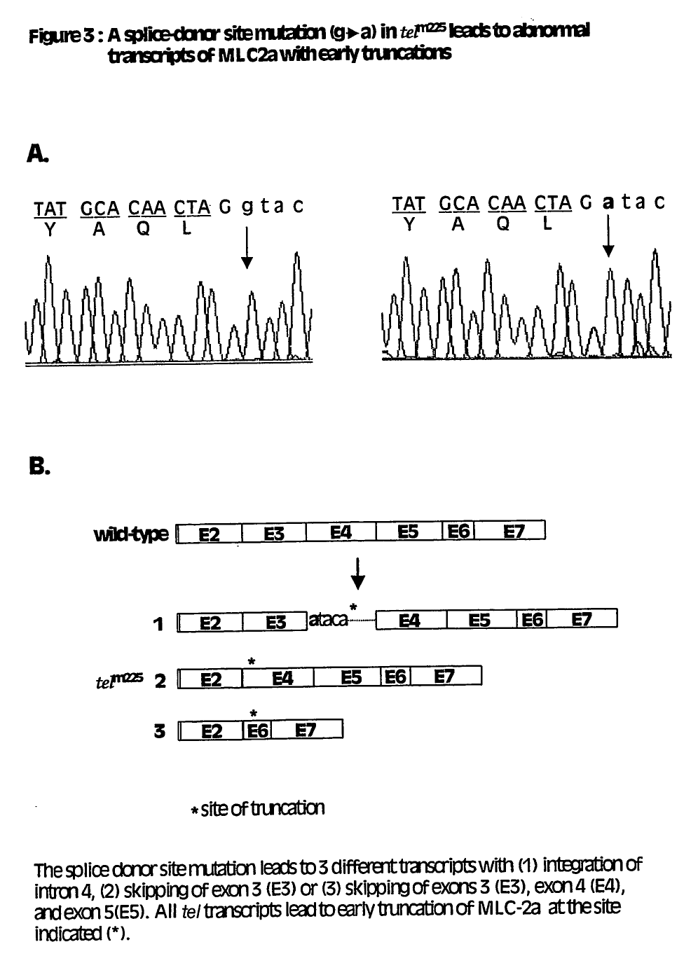

A BAC clone containing fb57f10 was isolated and shotgun sequenced. Four open reading frames were detected on this BAC, one encoding a highly conserved zebrafish regulatory myosin light chain gene (MLC-2a) (FIG. 2). Sequencing of candidate gDNA and cDNA revealed a splice donor site mutation in the zebrafish myosin light chain 2a (MLC-2a) gene (FIG. 3). Three different mutant splice products were detected in tel mutant cDNA, all transcripts, leading to premature termination of translation prior to exon 3 or 4, respectively (FIG. 3). Inhibition of zebrafish MLC-2a translation, using an antisense, morpholino-modified oligonucleotide (morpholino), generated with high penetrance a phenotype indistinguishable from tel mutant embryos. Zebrafish MLC-2a mRNA is expressed in cardiac precursor cells at 14 somites, and continues to be expressed throughout the heart until day 4 of development. Based on these data, we conclude that a loss of function mutation in zebrafish MLC-2a causes the tel phenotype. Two major myosin light chain 2 (MLC 2) isoforms are co-expressed in the early stages of vertebrate cardiogenesis, a cardiac ventricular isoform and a cardiac atrial isoform, each of which is tightly regulated in a muscle cell-type-specific manner during embryogenesis. Disruption of the myosin light chain 2v (MLC 2v) gene in mice leads to early embryonic death at embryonic day 12.5. Although in homozygote mutant ventricles upregulation of MLC-2a protein levels can be observed, the mice are displaying defects in sarcomeric assembly and an embryonic form of dilated cardiomyopathy characterized by a significantly reduced left ventricular ejection fraction. Point mutations in MLC-2v have been shown to be associated with a genetic form of human cardiomyopathy, and induction of the atrial MLC-2 isoform (MLC-2a) has been shown to occur in cardiac hypertrophy and failure. Our data demonstrate for the first time an essential role for MLC-2a both in ventricular and atrial contractility in vertebrate embryonic development. Analysis of the regulatory processes involved in re-expression of fetal genes during cardiac hypertrophy and/or heart failure can be used, according to the invention, in the identification of new therapeutic targets for these common diseases.

Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it is to be understood that it is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the appended claims.

1. A method of determining whether a test subject has, or is at risk of developing, a disease or condition related to Myosin Light Chain 2a, said method comprising analyzing a nucleic acid molecule of a sample from the test subject to determine whether the test subject has a mutation in a gene encoding said Myosin Light Chain 2a, wherein the presence of a mutation indicates that said test subject has, or is at risk of developing, a disease or condition related to Myosin Light Chain 2a.

2. The method of claim 1, wherein said test subject is a mammal.

3. The method of claim 1, wherein said test subject is a human.

4. The method of claim 1, wherein said disease or condition is a disease or condition of the heart.

5. The method of claim 4, wherein said disease or condition is heart failure.

6. A method for identifying a compound that can be used to treat or to prevent a disease or condition of the heart of associated with Myosin Light Chain 2a, said method comprising contacting an organism comprising a mutation in a gene encoding Myosin Light Chain 2a and having a phenotype characteristic of a disease or condition associated with Myosin Light Chain 2a with said compound, and determining the effect of said compound on said phenotype, wherein detection of an improvement in said phenotype indicates the identification of a compound that can be used to treat or to prevent said disease or condition of the heart.

7. The method of claim 6, wherein said disease or condition of the heart is heart failure.

8. The method of claim 6, wherein said organism is a zebrafish.

9. The method of claim 6, wherein said mutation in the gene encoding Myosin Light Chain 2a is the tell tale heart mutation.

10. A method of treating or preventing a disease or condition of the heart associated with Myosin Light Chain 2a in a patient, said method comprising administering to said patient a compound identified using the method of claim 6.

11. The method of claim 10, wherein said patient has a mutation in a gene encoding Myosin Light Chain 2a.

12. A method of treating or preventing a disease or condition associated with Myosin Light Chain 2a in a patient, said method comprising administering to said patient a functional Myosin Light Chain 2a protein or an expression vector comprising a nucleic acid molecule encoding said protein.

13. A substantially pure zebrafish Myosin Light Chain 2a polypeptide.

14. The polypeptide of claim 13, wherein said polypeptide comprises an amino acid sequence that is substantially identical to the amino acid sequence of SEQ ID NO:2.

15. The polypeptide of claim 13, wherein said polypeptide comprises the amino acid sequence of SEQ ID NO:2.

16. A substantially pure polypeptide comprising the sequence of SEQ ID NO:2 and variants thereof comprising sequences that are at least 95% identical to that of SEQ ID NO:2, and which have Myosin Light Chain 2a activity.

17. An isolated nucleic acid molecule comprising a sequence encoding a zebrafish Myosin Light Chain 2a polypeptide.

18. The nucleic acid molecule of claim 17, wherein said nucleic acid molecule encodes a polypeptide comprising an amino sequence that is substantially identical to the amino acid sequence of SEQ ID NO:2.

19. The nucleic acid molecule of claim 17, wherein said nucleic acid molecule encodes a polypeptide comprising the amino acid sequence of SEQ ID NO:2.

20. An isolated nucleic acid molecule that specifically hybridizes under high stringency conditions to the complement of the sequence set forth in SEQ ID NO: 1, wherein said nucleic acid molecule encodes a protein that has Myosin Light Chain 2a activity.

21. A vector comprising the nucleic acid molecule of claim 17.

22. A cell comprising the vector of claim 21.

23. A non-human transgenic animal comprising the nucleic acid molecule of claim 17.

24. The non-human transgenic animal of claim 23, wherein said animal is a zebrafish.

25. A non-human animal having a knockout mutation in one or both alleles encoding a Myosin Light Chain 2a polypeptide.

26. A cell from the non-human knockout animal of claim 25.

27. A non-human transgenic animal comprising a nucleic acid molecule encoding a mutant Myosin Light Chain 2a polypeptide.

28. The non-human transgenic animal of claim 27, wherein the non-human transgenic animal is a zebrafish.

29. The non-human transgenic animal of claim 27, wherein the non-human transgenic animal comprises the tell tale heart mutation.

30. An antibody that specifically binds to a Myosin Light Chain 2a polypeptide.

31. A method of modulating the activity of a Myosin Light Chain 2a polypeptide in a patient, said method comprising administering to the patient an RNA that stimulates or inhibits this activity.
